(19) United States
(12) Patent Application Publication
(10) Pub. No.: US 2026/0364150 A1
(43) Pub. Date: Nov. 19, 2026

(54) SKIN CELLULAR REGENERATION COMPOSITION AND METHOD

(71) Applicant: PI RD Max inc., Ayer's Cliff (CA)

(72) Inventor: Renaude Dube, Ayer's Cliff, QC (CA)

(21) Appl. No.: 19/477,308

(22) PCT Filed: Apr. 21, 2023
(86) PCT No.: PCT/CA2023/050545

§ 371 Date: Oct. 21, 2025

(51) Int. Cl. A61K 38/39; A61K 31/695; A61K 31/728; A61K 36/03; A61K 47/02

(52) U.S. Cl. CPC A61K 38/39; A61K 31/695; A61K 31/728; A61K 36/03; A61K 47/02

(57) ABSTRACT

A composition which comprise oxygenated water combined to other ingredients is provided. The composition is generally intended at helping treating damaged skin tissues and to percutaneously penetrate the skin of a user down to the dermis. More particularly, the composition for regenerating skin tissue may comprise between 80 to 95% W/W of oxygenated water and between more than 0 and 5% W/W of collagen. Also, the composition for regenerating skin tissue may comprise between 80 to 95% W/W of oxygenated water and between more than 0 and 5% W/W of elastin. A use of the composition for the treatment of damaged skin tissue is also provided.

### CROSS-REFERENCE TO RELATED APPLICATIONS

There are no cross-related applications.

### FIELD OF THE INVENTION

The present invention generally relates to compositions for regenerating skin cells and for methods of using said compositions. More specifically, the present invention relates to compositions for regenerating skin cells comprising oxygenated water with hydrolyzed collagen and/or elastin and uses.

### BACKGROUND OF THE INVENTION

When it comes to skincare for regeneration of skin tissues, specifically for treatment of scarred skin or ageing skin, traditional treatments often create a plurality of undesired secondary effects. As an example, silicone gels may be used to treat scars. The silicone gels typically create a silicone layer over the outer layer of the skin and therefore merely create a protective layer over the outer layer of the skin without treating damaged or aged tissues located under said outer layer of the skin.

Other formulations are also used to treat the skin, such as formulations comprising Retinol as an active ingredient. Such formulations may have beneficial effects such as cellular regeneration, exfoliation and synthesis of dermic collagen. However, such formulation may produce unwanted side effects such as desquamation, dry skin, pruritus and erythema.

Accordingly, there is a need for a composition and a method of use of such composition for overcoming the drawbacks of the prior art treatment formulation by providing deep skin cellular regeneration without the above-mentioned side effects.

### SUMMARY OF THE INVENTION

The aforesaid and other objectives of the present invention are realized by generally providing a composition for regenerating deep cells of the skin. The composition comprises between 80 and 95% W/W of oxygenated water and between more than 0 and 5% W/W of collagen.

The collagen may be any one or a combination of marine collagen powder, hydrolyzed collagen and hydrolyzed marine collagen. The composition may comprise between more than 0 and 5% W/W of organic silicon. The organic silicon may be Silanetriol. The composition may further comprise any one or a combination of Propandiol, Hyaluronic Acid, organic silicon, Soluble Collagen Peptides, Acrylates/C10-30 Alkyl Acrylate Crosspolymer, Benzyl Alcohol, Ethylhexylglycerin, Tocopherol, Coco-Caprylate/Caprate, Sodium Gluconate and Sodium Hydroxide. The composition may comprise 90.8% W/W of oxygenated water, 0.70% W/W of acrylates/C10-30 Alkyl Acrylate Crosspolymer, 0.75% W/W Sodium Hydroxide, 1.00% W/W Benzyl Alcohol and Ethylhexylglycerin and Tocopherol, 1.50% W/W Organic Silicon and Hyaluronic Acid, 0.20% W/W Sodium Gluconate, 2.00% W/W Propanediol, 2.00% W/W Sodium Hyaluronate, 1.00% W/W Aqua and Hydrolyzed Collagen and 0.05% W/W Coco-Caprylate/Caprate.

The composition may further comprise between more than 0 and 5% W/W of elastin. The composition may be a form selected from a gel, a cream, a liquid, a spray and a mask. The composition may be in the form of a cream and comprising between 20 and 25% W/W of oxygenated water.

In another aspect of the invention, a composition for regenerating deep cells of the skin is provided. The composition comprises between 80 and 95% V/V of oxygenated water and between more than 0 to 5% W/W of elastin.

The elastin may be hydrolyzed. The composition may further comprise between more than 0 and 5% W/W of a brown algae extract. The brown algae extract may be a Saccharina longicruris extract. The composition may further comprise any one or a combination of: Glycerin, Saccharina longicruris Extract, hydrolyzed Elastin, Sodium Hyaluronate, Acrylates/C10-30 Alkyl Acrylate Crosspolymer, Benzyl Alcohol, Ethylhexylglycerin, Tocopherol, Coco-Caprylate/Caprate, Sodium Gluconate and Sodium Hydroxide. The composition may comprise 91.8% W/W oxygenated water, 0.70% W/W Acrylates/C10-30 Alkyl Acrylate Crosspolymer, 0.75% W/W Sodium Hydroxide, 1.00% W/W Benzyl Alcohol and Ethylhexylglycerin and Tocopherol, 1.50% W/W Glycerin and Water and Laminaria saccharina Extract, 0.20% W/W Sodium Gluconate, 2.00% W/W Glycerin, 1.00% W/W Hydrolyzed Elastin, 1.00% W/W Sodium Hyaluronate and 0.05% W/W Coco-Caprylate/Caprate.

The composition may be in a form selected from a gel, a cream, a liquid, a spray and a mask. The composition may be in the form of a cream and comprising between 20 to 25% W/W of oxygenated water.

In another aspect of the invention, the use of any of the compositions as described above for regenerating deep skin tissue is provided. The use comprises applying the composition on the skin tissue to be treated. The use may further comprise applying the composition on the skin tissue following ablative laser, non-ablative laser, couperose laser and rosacea. The use may further comprise applying the composition on the skin tissue to be treated once per day during four consecutive days and twice per day during five consecutive days.

The features of the present invention which are believed to be novel are set forth with particularity in the appended claims.

### BRIEF DESCRIPTION OF THE DRAWINGS

The above and other objects, features and advantages of the invention will become more readily apparent from the following description, reference being made to the accompanying drawings in which:

FIG. 1 is an illustration of the effects of the use of a composition comprising collagen in accordance with the principles of the invention compared to a placebo composition showing the intensity of collagen I.

FIG. 2 is an illustration of the effects of the use of a composition comprising collagen in accordance with the principles of the invention compared to a placebo composition showing the intensity of collagen III.

FIG. 3 is an illustration of a graphic comparing the intensity of hyaluronic acids present in skin tissues following the use of a composition comprising collagen in accordance with the principles of the invention compared to a placebo composition.

FIG. 4 is an illustration of mature skin tissues having damaged and/or missing collagen and elastin.

FIG. 5 is an illustration of the use a composition in accordance with the principles of the invention on the mature skin tissues of FIG. 4.

FIG. 6 is an illustration of the effects of the use of the composition of FIG. 5 on the mature skin tissues.

FIG. 7 is an illustration of the effects of the use of a composition comprising collagen in accordance with the principles of the invention.

FIG. 8 is an illustration of the effects of the use of a composition comprising collagen in accordance with the principles of the invention.

FIG. 9 is an illustration of the effects of the use of a composition comprising collagen and a composition comprising elastin in accordance with the principles of the invention.

FIG. 10 is an illustration of the effects of the use of a composition comprising collagen in accordance with the principles of the invention.

### DETAILED DESCRIPTION OF THE PREFERRED EMBODIMENT

A novel skin cellular regeneration composition, method and use will be described hereinafter. Although the invention is described in terms of specific illustrative embodiment(s), it is to be understood that the embodiment(s) described herein are by way of example only and that the scope of the invention is not intended to be limited thereby.

Human skin generally acts as a protection barrier against external elements. To continue acting as a good barrier, the skin shall be provided with nutrients in order to maintain good health of the said skin. More importantly, the said nutrients shall penetrate deep layers of skins in order to maintain or reconstruct skin tissue layers down to the dermis and to promote communication between said skin tissue layers.

In some embodiments, a composition comprises oxygenated water combined to other ingredients. The oxygenated water generally helps the composition to percutaneously penetrate the skin of a user down to the dermis. The oxygenated water thus improves the transdermic power of the composition. To do so, oxygenated water comprises encapsuled oxygen nanobubbles found in a high concentration. Accordingly, the skin will better absorb the oxygenated water due to its oxygen content being higher than the ambient air's oxygen content. For example, the composition may comprise dissolved oxygen of over 50 mg/L in addition with forty-two million encapsulated oxygenated nanobubbles. The percutaneous penetration of oxygenated water generally aims at favoring the keratinocytes' epithelialization, increasing the angiogenesis process, stimulating connective tissue formation and/or increasing collagen presence in the skin and stimulating local immune response to accelerate healing of the skin. Transdermic penetration of the composition comprising oxygenated water contributes to the dermis and epidermis regeneration.

In an embodiment, the composition may further comprise collagen configured to penetrate the skin of a user. Collagen is the most abundant protein in the skin, and may represent up to 70% of the composition of the skin thereof. The collagen present in the skin absorbs water and plays a role in the resilience, cellular regeneration and wrinkles attenuation. Hence, the collagen that is present in the composition generally aims at increasing the amount of collagen in the skin, such as collagen I and III. The collagen may further stimulate hyaluronic acid and provide essential amino acids for the skin tissues. The essential amino acids may have a low molecular weight, such as between 3 and 6 Daltons.

The composition may comprise one or more types of collagens. As an example, the composition may comprise marine collagen, such as but not limited to marine collagen powder, hydrolyzed collagen, hydrolyzed marine collagen, or a combination thereof. Understandably, any other types of collagens originating from various sources and in various forms may be used.

The composition comprising hydrolyzed collagen, whether marine or not, may improve the interaction of the collagen with the skin tissues. The hydrolyzed collagen may have a low molecular weight and may comprise free amino acids, such as glycine, proline and hydroxyproline. As an example, hydrolyzed collagen may have a molecular weight (Mw) of 3-6 KDa, an isoelectric point (pl) of 3.68-5.7, a low viscosity and no film formation. Hydrolyzed collagen is thus vastly different than native collagen which may have a molecular weight (Mw) of 300 KDa, an isoelectric point (pl) of 7.0-8.3, a high viscosity and film formation.

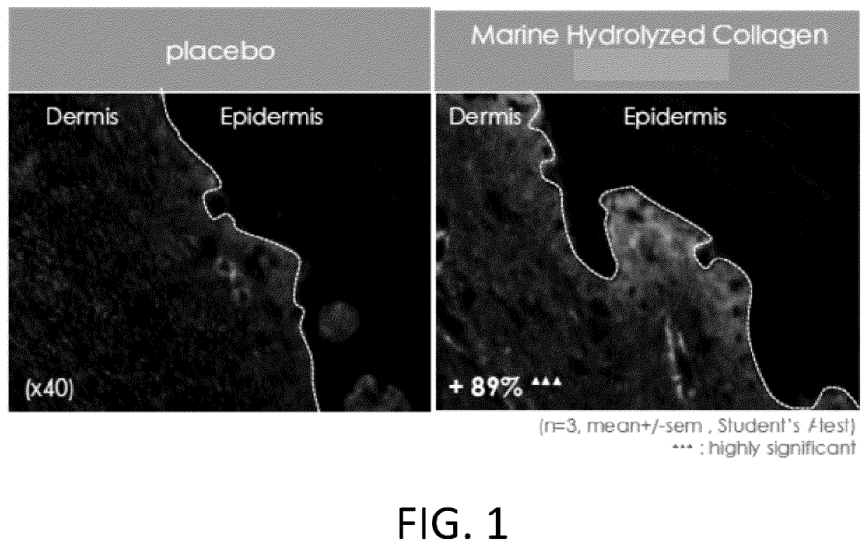

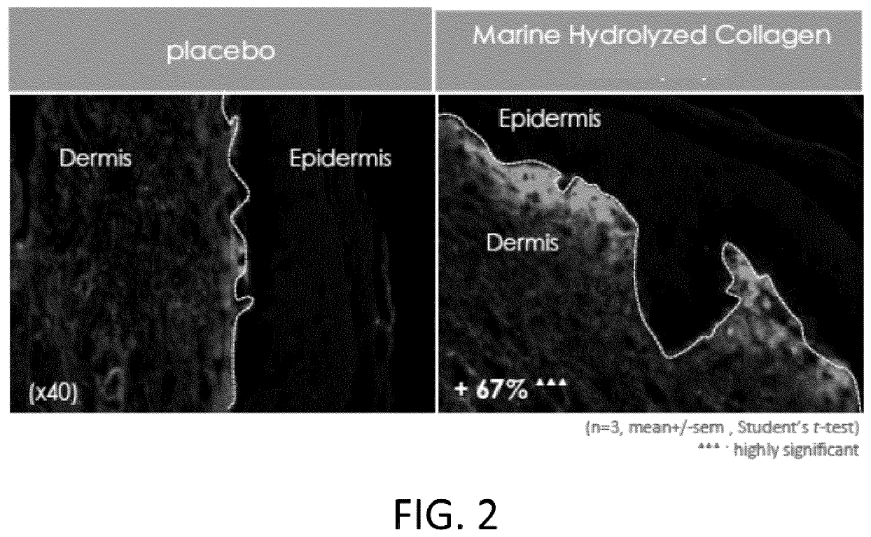

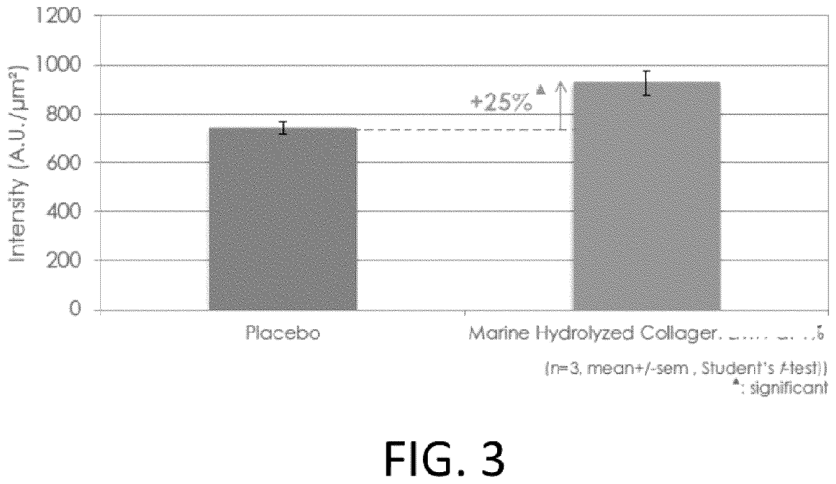

Referring to FIGS. 1 to 3, the effects of the use of a composition comprising hydrolyzed collagen in accordance with the principles of the present invention are shown. The tests use marine hydrolyzed collagen, however other types of collagens may also provide similar results. During the test, the composition comprising collagen has been applied twice per day during seventy-two (72) hours. FIGS. 1 and 2 show the results after using a placebo composition on the left and the composition comprising collagen on the right. FIG. 1 shows the presence of collagen I and FIG. 2 shows the presence of collagen II. It may be observed that the presence of collagen I and III is greatly increased compared to the placebo test results due to the collagen results being of a higher coloration intensity. FIG. 3 shows the intensity of hyaluronic acid in the skin tissues treated with the composition comprising collagen when compared to a placebo composition. It may be observed that the composition comprising collagen increased hyaluronic acid intensity by up to 25%. Accordingly, the tests which were performed clearly show that the presence of hydrolyzed collagen, such as marine hydrolyzed collagen, improves the intensity of collagen I and III and of hyaluronic acid in the skin.

The composition may further comprise additional compounds. The additional components may comprise one or more of propanediol, silanetriol, organic silicon and/or hyaluronic acid or a combination thereof. The additional compounds may aim at improving communication with the extra cellular matrix, increasing collagen I and III's synthesis, stimulating hyaluronic acid's synthesis, stimulating the fibroblasts and feeding the dermis. It may be appreciated that the presence of stem cells in the composition such as organic silicon, usually in combination with hyaluronic acid, may reduce the effects of stress on the skin, which further improves skin regeneration.

In an embodiment of the invention, the composition may comprise any one of the following compounds or a combination thereof: Oxygenated Water, Propandiol, Silantriol, Hyaluronic Acid, Hydrolyzed Collagen, Soluble Collagen Peptides, Acrylates/C10-30 Alkyl Acrylate Crosspolymer, Benzyl Alcohol, Ethylhexylglycerin, Tocopherol, Coco-Caprylate/Caprate, Sodium Gluconate and Sodium Hydroxide.

In yet another embodiment of the invention, an exemplary composition comprises the following compounds at the following concentrations:

| | Concentration |
|---|---|
| Compound | (% W/W) |
| Oxygenated Water | 90.8 |
| Acrylates/C10-30 Alkyl | 0.70 |
| Acrylate Crosspolymer | |
| Sodium Hydroxide | 0.75 |
| Benzyl Alcohol and | 1.00 |
| Ethylhexylglycerin and | |
| Tocopherol | |
| Organic Silicon and Hyaluronic | 1.50 |
| Acid | |
| Sodium Gluconate | 0.20 |
| Propanediol | 2.00 |
| Sodium Hyaluronate | 2.00 |
| Aqua and Hydrolyzed Collagen | 1.00 |
| Coco-Caprylate/Caprate | 0.05 |

In yet other embodiments, the above-described composition comprising collagen may be applied as a gel, a liquid, a topical cream or any other type of application medium.

In further embodiments, the composition may comprise the above-described compounds in a wider range of values. For example, the oxygenated water may be found between 80 to 95% W/W. In such embodiment, the composition may be a gel or the like. In other examples, the oxygenated water may be found between 20 to 30% W/W. In such embodiment, the composition may be a cream. It may be understood that by varying the weight value of the compounds in the composition, namely of the oxygenated water, it may be possible to further add other types of collagens to the composition. In some examples, the weight of marine collagen may vary between more than 0 to 5% W/W. Understandably, the compound comprising Silanetriol, organic silicon, and Hyaluronic Acid may further be found between more than 0 to 5% W/W.

In another embodiment, the composition may comprise elastin. Elastin is part of the family of structural proteins. Elastin may therefore be used to maintain the skin's density level, firmness and resilience. The elastin of the composition generally acts to regenerate the extra cellular matrix in the deep layers of the skin. Elastin may be used to increase density of the elastin fibers in the skin, to improve skin tone and help restructure the skin to increase density and firmness.

In some embodiments, the composition may further comprise brown algae, such as Saccharina longicruris, also called Laminaria saccharina. The algae may improve elasticity and density of the elastin fibers in the skin, may increase the hyaluronic acid levels in the skin and may improve that protection barrier of the skin.

In yet another embodiment of the invention, an exemplary composition comprising elastin comprises:

| | Concentration |
|---|---|
| Compound | (% W/W) |
| Oxygenated Water | 91.8 |
| Acrylates/C10-30 Alkyl | 0.70 |
| Acrylate Crosspolymer | |
| Sodium Hydroxide | 0.75 |
| Benzyl Alcohol and | 1.00 |
| Ethylhexylglycerin and | |
| Tocopherol | |
| Glycerin and Water and | 1.50 |
| Laminaria Saccharina Extract | |
| Sodium Gluconate | 0.20 |
| Glycerin | 2.00 |
| Hydrolyzed Elastin | 1.00 |
| Sodium Hyaluronate | 1.00 |
| Coco-Caprylate/Caprate | 0.05 |

In some embodiment, the composition may comprise any one of the following compounds or a combination thereof: Oxygenated Water, Glycerin, Saccharina longicruris Extract, Hydrolyzed Elastin, Sodium Hyaluronate, Acrylates/C10-30 Alkyl Acrylate Crosspolymer, Benzyl Alcohol, Ethylhexylglycerin, Tocopherol, Coco-Caprylate/Caprate, Sodium Gluconate and/or Sodium Hydroxide.

In further embodiments, the composition may comprise the above-described compounds in a wider range of values. For example, the oxygenated water may be found between 80 to 95% W/W. In such embodiment, the composition may be a gel or the like. In other examples, the oxygenated water may be found between 20 to 30% W/W. In such embodiment, the composition may be a cream.

It may be appreciated that the composition comprising elastin may also further comprise other types of elastin or collagen, such as marine collagen between more than 0 to 5% W/W. Understandably, the compound comprising Glycerin and Water and Laminaria Saccharina Extract may further be found between more than 0 to 5% W/W.

The composition may be comprised in a gel, a cream, a liquid, a spray, a mask or any other application medium known in the art of topical skin treatments. It may be appreciated that in compositions comprised in sprays or the like, the composition may comprise both hydrolyzed elastin and hydrolyzed collagen. The hydrolyzed elastin and hydrolyzed collagen may be found within the composition in any of the above-described ratios and the ratios of oxygenated water may be adjusted thereof.

The use of a skin cellular regeneration composition is also disclosed. In some embodiments, the use comprises using an embodiment of skin cellular regeneration composition as hereinabove described one time per day on a skin area to be treated. The use may further comprise using an embodiment of skin cellular regeneration composition until the treated skin's appearance is stabilized or improved.

The composition may be used for the treatment of scars, such as for post-surgical scars. The composition may further be used on skin following treatments such as ablative laser, non-ablative laser such as intense pulsed light (IPL) and neodymium-doped yttrium aluminum garnet (ND:YAG) laser, couperose laser and rosacea. In further embodiments, the use may comprise applying the composition immediately following a medical treatment or surgery on the area of skin which was treated or which had a surgery. The method further comprises applying the composition at least once a day for 0 to 4 days on the area of the skin which was treated or which had a surgery and applying at least twice a day for 5 days and more the composition on the said area of the skin until the skin is fully treated. Understandably, the composition may be used on damaged skin for any other reasons not listed hereabove.

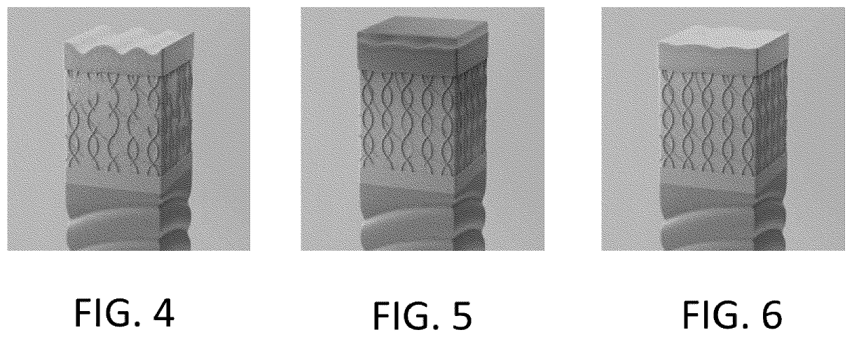

Referring now to FIGS. 4 to 6, the effects of the use of a composition of collagen and elastin as above described is illustrated. FIG. 4 shows mature skin tissues in which collagen and elastin of the tissues are damaged. FIG. 5 shows the deep penetration of the compositions when used on the mature skin tissue. FIG. 6 shows the subsequent effects of the use of the compositions on the mature skin tissues. It may be appreciated that the damaged skin tissues shown in FIG. 1 have been substantially repaired.

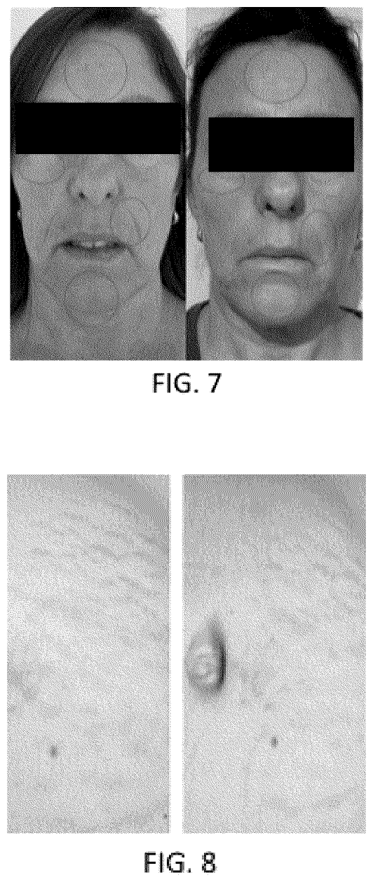

Referring now to FIG. 7, exemplary effects of use of an embodiment of the composition comprising collagen in accordance with the principles of the present invention are shown. In such an example, the composition has been used by the user twice a day for thirty days. On the left side of FIG. 7, the skin appearance of a user is shown before the use of the composition. As such, clear wrinkles may be observed in at least three identified locations on the user. On the right side of FIG. 7, the skin appearance of a user is shown after use of the composition for thirty consecutive days. The same locations as on the left side are shown and demonstrate reduced wrinkles visibility.

Referring now to FIG. 8, exemplary effects of use of an embodiment of a composition comprising collagen in accordance with the principles of the present invention are shown. In such an example, an embodiment of the composition was applied on the skin of a user twice a day during sixty consecutive days. On the left side of FIG. 8, the skin appearance of a user is shown before use of the composition on the said user. As shown, the skin of the user comprises stretch marks resulting from birth. On the right side of FIG. 8, the skin appearance of a user is shown after use of the composition. As shown, the skin of the user comprises an improve appearance of the stretch marks present before user. Namely, the stretch marks size and/or numbers have been reduced.

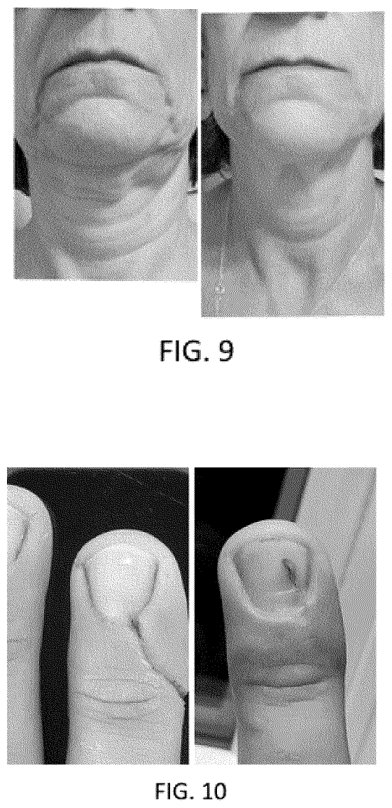

Referring now to FIG. 9, exemplary effects of use of an embodiment of two compositions in accordance with the principles of the present invention are shown. A first of the compositions comprises collagen and a second of the compositions comprises elastin. The first and second compositions have been applied on the skin of a user twice a day during twenty-eight consecutive days. Comparing the left and right sides of the illustration representing before and after the use of the compositions, it may be observed that the skin tissue in the neck area of the user where the compositions have been applied have become much firmer and thus the skin tissues are denser.

Referring now to FIG. 10, exemplary effects of use of an embodiment of a composition comprising collagen in accordance with the principles of the present invention are shown. The composition has been applied on the skin of a user twice a day during fourteen consecutive days. On the left side of the FIG. 10, the skin appearance of a user is shown prior use of the composition on the said user. As shown, the user had a skin injury. The skin injury is a clear-cut mark. On the right side of FIG. 10, the skin appearance of the user is shown after use of the composition on the skin injury. As shown, it may be observed that the cut mark on the skin has been cicatrized and is barely visible anymore.

While illustrative and presently preferred embodiment(s) of the invention have been described in detail hereinabove, it is to be understood that the inventive concepts may be otherwise variously embodied and employed and that the appended claims are intended to be construed to include such variations except insofar as limited by the prior art.

1) A composition for regenerating deep cells of the skin comprising:
between 80 and 95% W/W of oxygenated water; and
between more than 0 and 5% W/W of collagen.

2) The composition of claim 1, the collagen being any one or a combination of marine collagen powder, hydrolyzed collagen and hydrolyzed marine collagen.

3) The composition of claim 1, further comprising between more than 0 and 5% W/W of organic silicon.

4) The composition of claim 3, the organic silicon being Silanetriol.

5) The composition of claim 1, further comprising any one or a combination of: Propandiol, Hyaluronic Acid, organic silicon, Soluble Collagen Peptides, Acrylates/C10-30 Alkyl Acrylate Crosspolymer, Benzyl Alcohol, Ethylhexylglycerin, Tocopherol, Coco-Caprylate/Caprate, Sodium Gluconate and Sodium Hydroxide.

6) The composition of claim 5 comprising:
90.8% W/W of oxygenated water;
0.70% W/W of acrylates/C10-30 Alkyl Acrylate Crosspolymer;
0.75% W/W Sodium Hydroxide;
1.00% W/W Benzyl Alcohol and Ethylhexylglycerin and Tocopherol;
1.50% W/W Organic Silicon and Hyaluronic Acid;
0.20% W/W Sodium Gluconate;
2.00% W/W Propanediol;
2.00% W/W Sodium Hyaluronate;
1.00% W/W Aqua and Hydrolyzed Collagen; and
0.05% W/W Coco-Caprylate/Caprate.

7) The composition of claim 1 further comprising between more than 0 and 5% W/W of elastin.

8) The composition of claim 1, the composition being in a form selected from a gel, a cream, a liquid, a spray and a mask.

9) The composition of claim 1, the composition being in the form of a cream and comprising between 20 and 25% W/W of oxygenated water.

10) A composition for regenerating deep cells of the skin comprising between 80 and 95% V/V of oxygenated water and between more than 0 to 5% W/W of elastin.

11) The composition of claim 10, the elastin being hydrolyzed.

12) The composition of claim 10, further comprising between more than 0 and 5% W/W of a brown algae extract.

13) The composition of claim 12, the brown algae extract being a Saccharina longicruris extract.

14) The composition of claim 10, further comprising any one or a combination of: Glycerin, Saccharina longicruris Extract, hydrolyzed Elastin, Sodium Hyaluronate, Acrylates/C10-30 Alkyl Acrylate Crosspolymer, Benzyl Alcohol, Ethylhexylglycerin, Tocopherol, Coco-Caprylate/Caprate, Sodium Gluconate and Sodium Hydroxide.

15) The composition of claim 14 comprising:
91.8% W/W oxygenated water;
0.70% W/W Acrylates/C10-30 Alkyl Acrylate Crosspolymer;
0.75% W/W Sodium Hydroxide;
1.00% W/W Benzyl Alcohol and Ethylhexylglycerin and Tocopherol;
1.50% W/W Glycerin and Water and Laminaria saccharina Extract;
0.20% W/W Sodium Gluconate;
2.00% W/W Glycerin;
1.00% W/W Hydrolyzed Elastin;
1.00% W/W Sodium Hyaluronate; and
0.05% W/W Coco-Caprylate/Caprate.

16) The composition of claim 10 being in a form selected from a gel, a cream, a liquid, a spray and a mask.

17) The composition of claim 10, the composition being in the form of a cream and comprising between 20 to 25% W/W of oxygenated water.

18) The use of a composition for regenerating deep skin tissue according to claim 1 comprising applying the composition on the skin tissue to be treated.

19) The use of claim 18 further comprising applying the composition on the skin tissue following ablative laser, non-ablative laser, couperose laser and rosacea.

20) The use of claim 18, comprising applying the composition on the skin tissue to be treated once per day during four consecutive days and twice per day during five consecutive days.

21) The use of a composition for regenerating deep skin tissue according to claim 10 comprising applying the composition on the skin tissue to be treated.

22) The use of claim 21 further comprising applying the composition on the skin tissue following ablative laser, non-ablative laser, couperose laser and rosacea.

23) The use of claim 22, comprising applying the composition on the skin tissue to be treated once per day during four consecutive days and twice per day during five consecutive days.
